(19) United States
(12) Patent Application Publication
(10) Pub. No.: US 2006/0052947 A1
(43) Pub. Date: Mar. 9, 2006

(54) Biofabrication of transistors including field effect transistors

(76) Inventor: Evelyn Hu, Santa Barbara, CA (US)

Correspondence Address: FOLEY AND LARDNER LLP;SUITE 500, 3000 K STREET NW, WASHINGTON, DC, 20007, US

(21) Appl. No.: 11/130,399

(22) Filed: May 17, 2005

Related U.S. Application Data
(60) Provisional application No. 60/571,532, filed on May 17, 2004.

(51) Int. Cl. H01L 21/00; G06F 19/00; H01L 29/08; G01N 33/50

(52) U.S. Cl. 702/20; 438/1; 257/40; 332/128

(57) ABSTRACT

Use of peptides and other biological agents for fabrication of transistors, field effect transistors, and components thereof. An intermediate component for use in fabrication of a field effect transistor, the component comprising at least two of the following transistor elements: (i) source, (ii) drain, (iii) channel, (iv) gate, and (v) dielectric, wherein the at least two elements are combined by a biological agent comprising at least two binding structures, wherein each of the binding structures is bound to one of the at least two elements. The channel can be a nanowire or a nanotube which is surrounded by a high-K dielectric material, which is further surrounded by a metal gate layer. The biological agent can be a bifunctional peptide which binds dielectric to channel or binds dielectric to gate materials.

This application claims priority to provisional patent application Ser. No. 60/571,532 to Hu filed May 17, 2004, which is hereby incorporated by reference in its entirety.

### BACKGROUND

The scientific and commercial pressure to shrink the size and decrease the switching times of semiconductor devices and memories continues and is now an active area for nanotechnology research and development. Miniaturization of electronic devices can be viewed as progressively going through a series of successfully smaller stages including: small-scale integration (SSI), medium-scale integration (MSI), large-scale integration (LSI), very-large-scale integration (VLSI), ultra-high-scale integration (UHSI), and molecular electronics. “Roadmaps” from one international organization, (International Technology Roadmap for Semiconductors, ITRS), have set clear specifications in the form of “nodes” for the gate length and feature spacing of these circuits projecting out more than a decade. The technology roadmap is geared toward further miniaturizing CMOS technology (complementary—metal oxide semiconductor). The goal is to extend Moore's law into the future and delay its end. Recognized node sizes include 250, 180, 130, 90, 65, 45, 32, and 22 nm for nominal feature sizes.

In particular, a present need exists to make better, smaller, and faster nanostructured devices for digital integrated circuits including field effect transistors (FETs). One dimensional nanostructures including nanowires and nanotubes are recognized as an important tool for miniaturizing metal oxide semiconductor field effect transistors (MOSFETs). The small dimensions, however, mean that new approaches are needed to solve technical problems. For example, formidable technical challenges with putting nanowires into MOSFET devices include, for example, (i) placing and connecting the nanowires and nanotubes onto a device including transistor and MOSFET devices, (ii) controlling the nanowire length and layer dimensions, and (iii) using new materials other than Si and SiOₓ, as the insulator or dielectric layer or creating additional gate material layers surrounding other gate layers.

More specifically, the challenge faced by the ITRS program is to try to maintain the same high quality device operation as seen in long channel devices (about 100-200 nm), while scaling down devices to short channels (about 20-50 nm). In order for a smaller footprint gate electrode to switch a sufficiently large volume of the channel region into inversion, the insulation layer thickness must also decrease. This is an important issue. Currently, SiO₂ insulator layers are approaching 4 atomic layers in thickness. At these thicknesses, it becomes much harder to control electrical breakdown across the insulator or leakage current through defects. Additionally, decreasing the size of an electrode can increase “edge effects.” In this case, this is referring to the capacitive coupling between gate to drain and gate to source mediated by electrical fields from the edges of these two regions as compared to the coupling of gate to channel regions via perpendicular electric fields. Besides impacting the efficiency of the MOSFET, these scale dependent geometrical effects also introduce opportunities for device performance variability.

One unconventional approach to solving these problems is to borrow and learn from nature including using the concepts of self-assembly and selective interactions and selective binding. Biological systems such as viruses, proteins, or peptides traditionally are not generally associated with non-biological commerce such as transistors including PETS. However, biological systems have been recognized which can selectively bind to inorganic, including semiconductor, crystal structures as well as other useful material structures. Moreover, biological systems have been recognized which can catalyze formation of and nucleate inorganic nanoparticles and nanocrystals. Biological systems can bind to preexisting nanoparticles and nanocrystals and assemble them. The biological systems can be to some extent synthetic or engineered, providing exquisite control over the inorganic material which can be difficult if not impossible to achieve by other methods. Hence, a need exists to better employ the methods and materials of biology and self assembly to fuel the drive to miniaturize.

### SUMMARY

The present invention provides a variety of platform capabilities to provide better transistor devices including field effect transistors and MOSFETs including:
(a) use of selective biological agents or molecules including synthetic or engineered peptides to enable growth of one material around another when constructing multi-layered nanowires or nanotubes (for example, growth of high k dielectric material HfO₂ around Si);
(b) use of selective biological agents or molecules, including synthetic or engineered peptides, to bind a specific region such as the end of a nanowire or nanotuhe device to another region on a planar circuit;
(c) use of biological agents as templates, including viral templates, to grow or assemble non-uniform semiconductor nanowires or nanotubes of controlled length and with specific end groups for nanowire/nanotube location or materials growth.

In one embodiment, the invention provides an intermediate component for use in fabrication of a field effect transistor, the component comprising at least two of the following transistor elements: (i) source, (ii) drain, (iii) channel, (iv) gate, and (v) dielectric, wherein the at least two elements are combined by a biological agent comprising at least two binding structures, wherein each of the binding structures is bound to one of the at least two elements. In particular, embodiments are preferred wherein the biological agent functions to bind the channel to the dielectric, or bind the dielectric to the gate. The biological agent comprising the binding structures can exhibit different levels of binding strength and specificity, and the two binding structures do not need to have the same level of binding specificity and binding strength.

Also provided is an intermediate component for fabricating a metal oxide semiconductor field effect transistor (MOSFET), the component comprising at least two of the following field effect transistor elements: (i) source, (ii) drain, (iii) channel, (iv) gate, and (v) dielectric, wherein the at least two elements are combined by a biological agent comprising at least two binding structures, wherein each of the binding structures is bound to one of the at least two elements.

Also provided is an intermediate component for fabricating a field effect transistor, the component comprising at least the channel element and the dielectric element, wherein the channel and dielectric elements are combined by a biological agent comprising at least two binding structures, wherein each of the binding structures is bound to the channel and dielectric elements.

Also provided is an intermediate component for fabricating a field effect transistor, the component comprising at least the gate element and the dielectric element, wherein the gate and dielectric elements are combined by a biological agent comprising at least two binding structures, wherein each of the binding structures is bound to the gate and dielectric elements.

Also provided is an intermediate component for fabricating a field effect transistor, the component comprising at least the channel element and the source or drain element, wherein the channel and source or drain elements are combined by a biological agent comprising at least two binding structures, wherein each of the binding structures is bound to the channel and the source or drain elements.

Also provided is an electronic device comprising a plurality of field effect transistors, wherein the field effect transistors comprise channels comprising nanowires which are substantially monodisperse in length.

Also provided is an integrated circuit comprising a plurality of field effect transistors, wherein the field effect transistors comprise channels comprising nanowires which are substantially monodisperse in length.

Also provided is an electronic device comprising a plurality of metal oxide semiconductor field effect transistors (MOSFETS), wherein the field effect transistors comprise channels comprising nanowires which are substantially monodisperse in length.

Also provided is a nanowire structure comprising a nanowire core and a first nanowire outer layer surrounding the core, wherein a biological agent comprising at least two binding structures is used to combine the nanowire core and the nanowire outer layer.

Also provided is an intermediate component for use in fabrication of a transistor, the component comprising at least two of the following transistor elements: (i) source, (ii) drain, (iii) channel, (iv) gate, and (v) dielectric, wherein the at least two elements are combined by a biological agent comprising at least two binding structures, wherein each of the binding structures is bound to one of the at least two elements.

Also provided is a process for fabrication of elements of a field effect transistor comprising a source, a drain, a channel, a gate, and a dielectric as elements, comprising the step of combining at least two of the elements, wherein at least one biological agent comprising at least two binding structures is used to combine the at least two elements.

Also provided is a process for fabricating elements of a metal oxide semiconductor field effect transistor (MOSFET) comprising a source, a drain, a channel, a gate, and a dielectric as elements, comprising the step of combining at least two of the elements, wherein at least one biological agent comprising at least two binding structures is used to combine the at least two elements.

Also provided is a process for fabricating elements of a field effect transistor, comprising the step of combining at least a channel element and a dielectric element, wherein at least one biological agent comprising at least two binding structures is used to combine the channel and dielectric elements.

Also provided is a process for fabricating elements of a field effect transistor, comprising the step of combining at least a gate element and a dielectric element, wherein at least one biological agent comprising at least two binding structures is used to combine the gate and dielectric elements.

Also provided is a process for fabricating elements of a field effect transistor, comprising the step of combining at least a channel element and a source or drain element, wherein at least one biological agent comprising at least two binding structures is used to combine the channel element and the source or drain element.

Also provided is use of a biological binding agent to assemble elements of a field effect transistor.

Also provided is use of a peptide binding agent to assemble elements of a field effect transistor.

Also provided is a method for engineering the surface of a nanowire with an outer layer material comprising the step of binding the surface of the nanowire with a biological agent comprising at least two binding structures, one binding structure for the surface, and one binding structure for the outer layer material.

Also provided is a biological agent represented by A-B-C, wherein A and C are selective binding structures and B is an optional linking structure, wherein A and C selectively bind to a channel, a dielectric, a gate, a source, or a drain material.

Also provided is a component for use in fabrication of a field effect transistor, the component comprising at least two of the following transistor elements: (i) source, (ii) drain, (iii) channel, (iv) gate, and (v) dielectric, wherein the at least two elements are combined by a biological agent comprising at least two binding structures, wherein each of the binding structures is bound to one of the at least two elements.

Also provided is a field effect transistor comprising a nanowire or nanotube channel, a high-K dielectric material surrounding the channel, and a metal layer surrounding the high-K dielectric material.

Also provided is a transistor comprising a nanowire or nanotube channel, a dielectric material surround the channel and having a K value of about 10 or more, and a gate layer surrounding the dielectric material.

Also provided is a method of forming a dielectric layer surrounding a nanowire or a nanotube comprising the steps of providing the nanowire or nanotube, providing the dielectric material or a precursor thereof, providing a biological agent which comprises at least two binding structures, and forming the dielectric layer on the nanowire or nanotube in the presence of the biological agent.

Also provided is a method of forming a gate layer surrounding a dielectric material comprising the steps of providing the dielectric material, providing the gate material or a precursor thereof, providing a biological agent which comprises at least two binding structures, and forming the gate layer on the dielectric material in the presence of the biological agent.

Also provided is a method of forming a connection between a nanowire or a nanotube and a source or a drain, comprising the steps of providing a biological agent which comprises at least two binding structures, providing the nanowire or the nanotube, providing the source or drain, and connecting the nanowire or nanotube with the source or drain in the presence of the biological agent.

Advantages of the present invention include MOS transistor fabrication technology that, for example: (1) incorporates nanowire or nanotube transistors of controllable dimensions; (2) allows controlled formation of gate dielectrics of a variety of compositions, (3) allow the controlled formation of gate materials overlying the gate dielectric, (4) represents a massively parallel fabrication of transistor components, and (5) allows for selective attachment of the transistors onto the appropriate sites in the circuit.

### BRIEF DESCRIPTION OF THE DRAWINGS

FIG. 1 provides an illustrative example of a field effect transistor.

FIG. 2 illustrates the evolution from planar MOSFET geometry to “wrap around gate” geometry.

FIG. 3 illustrates engineering of nanowires (top) and filamentous bacteriophage (bottom).

FIG. 4 illustrates phage-mediated templating and assembly.

### DETAILED DESCRIPTION

Priority provisional patent application, Ser. No. 60,571,532 to Hu filed May 17, 2004 is hereby incorporated by reference in its entirety.

### Use of Biological Agent for Biofabrication of Transistors, Field Effect Transistors, and their Components

The present invention provides a variety of embodiments, wherein transistors, field effect transistors, and their components are assembled in part by biofabrication making use of biological agents, including biological peptide agents, which are described further below. In a preferred embodiment, a biological agent is used both to form a nanowire or a nanotube, which can be used to form a transistor component such as a channel, and then is also used to fabricate other transistor parts such as the dielectric or the gate. In addition, the biological agent can be used to bind the transistor, or components thereof, to other circuit parts in an integration process.

In one embodiment, the present invention provides an intermediate component for fabricating or assembling a transistor such as a field effect transistor, wherein the component comprises at least two of the following field effect transistor elements: (i) source, (ii) drain, (iii) channel, (iv) gate, and (v) dielectric, wherein the at least two elements are combined by a biological agent comprising at least two binding structures, wherein each of the binding structures is bound to one of the two elements. In preferred embodiments, the biological agent comprises peptide which is described further below.

In another embodiment, the present invention also provides a process for fabrication of an intermediate component for fabricating or assembling a transistor such as a field effect transistor, wherein the component comprises at least two of the following transistor elements: (i) source, (ii) drain, (iii) channel, (iv) gate, and (v) dielectric, wherein the at least two elements are combined by a biological agent comprising at least two binding structures, wherein each of the binding structures is bound to one of the two elements. In preferred embodiments, the biological agent comprises peptide which is described further below.

In practicing the invention, one does not have to select all of these elements but a biological agent is used to combine at least two of them. For example, the two elements can be the channel and the dielectric. Alternatively, the two elements can be the channel and the source, or the channel and the drain. Alternatively, the two elements can be the dielectric and the gate. Some transistor designs do not entail use of a dielectric.

In a preferred embodiment, the channel can be a one dimensional structure with nanoscopic dimensions including a nanowire or a nanotube.

The invention provides final devices, components, and intermediate components for fabrication and assembly with particular emphasis on various kinds of transistors, field effect transistors, and MOSFETs. See, for example, Campbell, Science and Engineering of Microelectronic Fabrication, 2nd Ed., Oxford Press, 2001 (chapter 16, for example, on CMOS). Other types of field effect transistors include NMOS, PMOS, MISFETs, MESFETs, JFETs, bipolar transistors, and hybrid/power transistors. High speed transistors including TeraHertz transistors are within the scope of the invention. Crossed nanowire FETs are within the scope of this invention.

Intermediate components can be fabricated, and then subjected to further fabrication steps to prepare a final product.

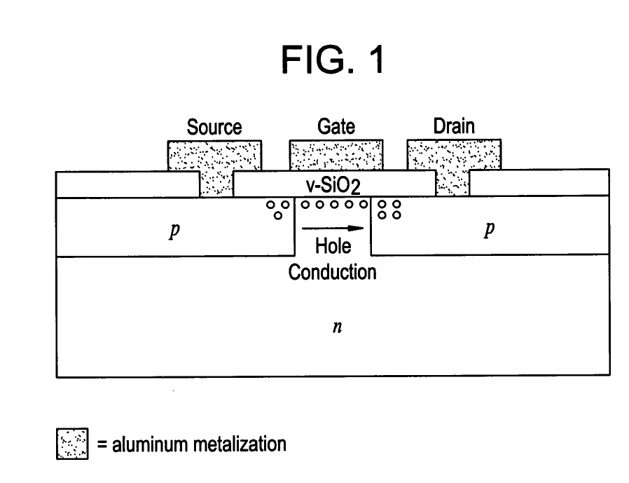

Field effect transistors, a subject of the preferred embodiment of the present invention, are important solid-state devices, and FETs have been developed that comprise nanowires and nanotubes. Materials used in semiconductor technology, including transistors, can be found in, for example, Chapter 12 and other chapters in Introduction to Materials Science for Engineers, (4^{th} Ed.), by J. S. Shackelford (1996). Various types of FETs are described generally in Chapter 8 of Solid State Electronic Devices, (4^{th} Ed), by B. G. Streetman (1995). FIG. 1 provides illustrative embodiments for traditional FETs. The FET can be of a p-type and n-type semiconductor and can comprise a source, drain, channel, dielectric, and gate as fundamental elements. Voltage can be applied to the gate which can control the conductivity and can make the channel conductive and provide a current flow from the source to the drain. Removal of the voltage on the gate effectively stops the overall current.

More specifically, and for purposes of understanding and practicing the present invention, the source (S) and drain (D) regions generally are highly doped, thus containing a high density of carriers and are capable of carrying high current. In an enhancement mode MOSFET, (the most common type) the channel region, below the gate has the same low doping as the bulk substrate and therefore conducts poorly, thus ohmically isolating the source and drain. When the gate voltage is moved with respect to the bulk substrate, however, an inversion region is created within the channel, where carrier density is increased and the MOSFET now draws current between source and drain. It is important generally that gate voltages result in efficient modulation of a large part of the channel region without causing any direct current to flow from the gate electrode to the substrate via leakage current or electrical breakdown across the insulation layer just below the gate. Additionally, gate voltage signal should have low coupling (ohmic or capacitive) to the source or drain regions or electrodes. More importantly these various coupling factors should be constant from device to device for proper operation of ICs.

In the present invention, the FETs can be prepared with use of different and new materials as a result of or necessitated by miniaturization. Use of the biological agents in the present invention can enable use of new materials. As lateral device dimensions are scaled down, vertical dimensions (such as the thickness of gate oxides, depths of source and drain doping profiles) generally are commensurately scaled to maintain good device performance. As gate dielectrics then are scaled down to dimensions on the order of a nanometer, issues of dielectric breakdown and gate leakage become paramount. These issues have resulted in a change in gate dielectric from traditional SiO₂ to potentially more robust high K dielectrics, such as HfO₂ and SrO. Recent changes also include returning to metals as a gate material over the common polycrystalline silicon (poly-silicon) choice as electron densities can be much higher in metals. All of these changes contribute to keep switching performance high while gate lengths are reduced.

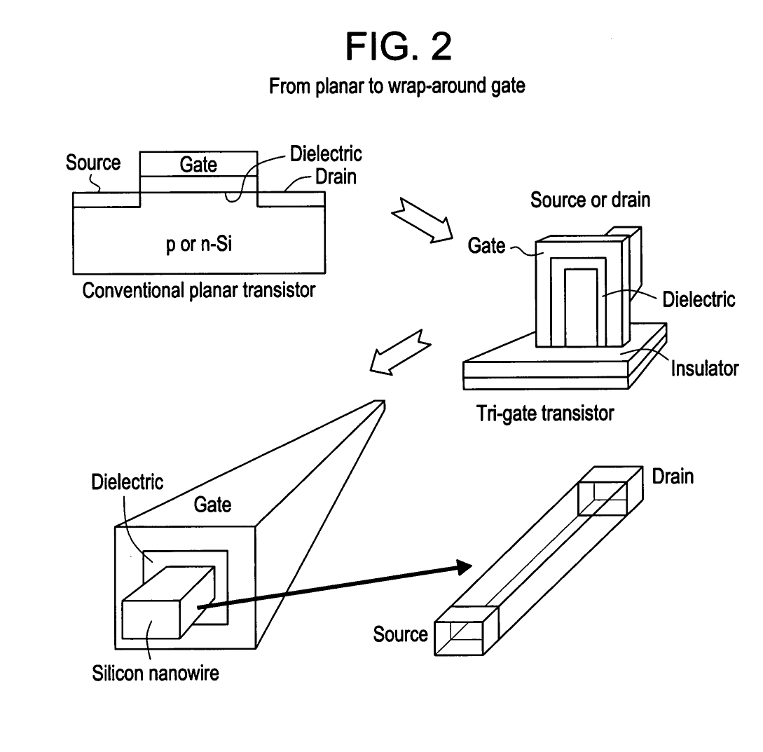

In the present invention, the FET geometry can also change as a result of miniaturization in addition to the changes in the material selection. For example, even if the materials issues are satisfactorily addressed, as the gate length is scaled well below 50 nm, unacceptable gate leakage and poor modulation may be a necessary consequence of the planar transistor technology. Recent successful solutions to this problem include the FinFET: a non-planar approach, in which the source-drain current is carried through a thin, silicon ‘fin’ that is controlled by a double gate. A tri-gate embodiment represents a subsequent development: the green silicon ‘fin’ of the FinFET is shortened to more closely resemble a ‘wire’ of silicon, and full modulation of the current is obtained by a tri-gate, or a gate that wraps around the Channel Region on three sides. FIG. 2 illustrates this evolution from planar MOSFET geometry to “wrap around gate” geometry. The improved ratio of electric field coupling from gate to channel regions vs. gate to source or drain regions by moving to a cylindrical geometry is best visualized by considering the electric field lines generated by each configuration. This is the same mechanism that allows cylindrical magnetic coils or transformers to have high central field intensity vs. fringe fields. The result can be much more dramatic control over channel region conductivity from a very small gate charge.

As the semiconductor industry moves towards a “wrap around gate” MOSFET design, which is part of the present invention, it is important to keep in mind the fundamental performance constraints that can be met by successful implementation of the present invention.
i) Strong gate coupling to channel region; weak gate coupling to source and drain
ii) Uniform and repeatable interface between gate and channel (low trap charged states)
iii) High breakdown, low leakage current across insulator layer
iv) Tight dimensional and electrical characteristics tolerances.

Additional performance parameters include the equivalent oxide thickness (EOT) and capacitive effective thickness (CET), and high-K dielectricimetal gate systems can be used to achieve better EOT and CET parameters. In general, EOT less than, for example, about 2 nm, and more particularly less than about 1 nm are desired.

The FinFet and TriGate designs are shown in FIG. 2 which can be adapted for purposes of the present invention. Fabricating these devices involves significant advances in lithographic fabrication steps which can be used and improved upon in the practice of the present invention. For example, semiconductor, insulator and gate materials can be layered not only in a direction perpendicular to the substrate, as is typical, but also in two directions parallel to the substrate plane. High aspect ratio structures are needed, and very well defined photoresist features should be controlled. High resolution microlithography and nanolithography processes can be used to practice the present invention including, for example, EUVL and nanoimprint lithography.

Typical FETs in the present invention can be characterized by a gate length or gate width which should be as small as possible to provide miniaturization. FETs can be based on Si or GaAs, but GaAs can be higher in cost and be more difficult to process. It also can be based on oxide.

Patent literature noting both field effect transistors and nanowires or nanotubes include, for example, U.S. Pat. Nos. 5,607,876; 5,962,863; 6,159,856; 6,256,767; 6,559,468; 6,602,974; and 6,709,929. Many patents and patent publications on field effect transistors are assigned to Intel including, for example, U.S. Pat. Nos. 6,734,498; 6,716,046; 6,707,120; 6,689,702; 6,605,845; 6,570,220; and 6,528,856. See also, U.S. patent publications 2004/0065903; 2003/0146479; 2003/0119248; 2003/0052333; 2003/0020075; and 2003/0015737. U.S. Patents which note or describe wrap around gate technology include, for example, U.S. Pat. Nos. 6,709,929; 6,664,143; 6,649,959; 6,649,935; 6,440,801; 6,413,802; 6,358,791; 6,355,520; 6,114,725; 6,034,389; 5,780,327; and 5,689,127. U.S. published patent applications which describe biofabrication, transistor, and field effect transistor technology include 2002/0171079, published Nov. 21, 2002 to Braun et al. (see FIG. 12 for a field effect transistor); 2001/0044114 to Connoly published Nov. 22, 2001; and 2004/0058457 published Mar. 25, 2004 to Huang et al (including description of bifunctional peptides), which are hereby incorporated by reference in their entirety.

### One Dimensional Nanowires and Nanotubes Generally

In general, the one dimensional structures forming the channel can be nanowires or nanotubes. The shape of the one dimensional structure, including geometry, length, and width, and the composition and electrical properties can be adapted for use in a channel, and for use in the nanostructured transistors of the invention. The one dimensional structures can be solid structures or have openings including tubular structures and porous structures. They can have opening at the end and openings which traverse the length or width of the one-dimensional structure. They can be considered rods and can have aspect ratios which are controlled to provide the desired properties. For example, aspect ratio can be, for example, about 50 or less, or about 25 or less, or about 10 or less. The one dimensional structures can be made of any materials which can function as a transistor channel and for field effect transistors provide the inversion properties, or other properties, to provide for current on/off effects. High mobility channels are generally desired. They can be made of traditional semiconductive elements or of polymeric materials such as electronically conductive polymers. Nanotubes and nanowires can be used in parallel wherein a plurality of channels are in parallel with each other, and the source and drains at the end of the channel are aligned. The plurality of parallel channels can be modulated together in sync or differently out of sync. The channel can float.

One step of the present invention is providing nanowires and nanotubes, be they chemically fabricated nanowires or nanotubes or provided by biological synthesis of nanowires or nanotubes. Carbon nanotubes can be used.

The nanowires or nanotubes can be homogeneous or heterogeneous. They can be crystalline. The nanowires can comprise inorganic elements, including semiconducting elements such as silicon. The nanotubes can be carbon nanotubes. Nanotubes can be reacted to provide elements other than carbon. Other elements can be on the surface of the nanotube or on the interior.

Each end of the nanowire or nanotube can be modified. This allows the one dimensional structure to be connected to source or drain. Modulated structures can be fabricated comprising dissimilar segments. For example, nanowires comprising dissimilar segments can comprise GaAs, GaP, Si, Si/Ge, InP, InAs, and other semiconductive segments. Alternating P and N regions can be prepared. Moreover, the nanowires and nanotubes can comprise compositional superlattices, wherein the composition varies along the length of the nanowire or nanotube. The nanowires can comprise core-shell heterostructures. Multiple shells can be prepared surrounding a core. Hence, a nanowire can be represented by [X-Y]n wherein n represents repeating units of different compositions X and Y. Nanowires can also be represented by S-C-D structures wherein S is the source, C is the channel, and D is the drain.

In a preferred embodiment, nanowires are formed by fusing adjacent nanoparticles or nanocrystals including semiconductive nanocrystals or quantum dots. Fusing can be carried out by heat treatment and templates can be used to position the nanoparticle or nanocrystals to be adjacent to each other in a linear fashion.

The nanowire or nanotube can be crystalline, semicrystalline, or amorphous as long as it can function as a channel. The nanowire or nanotube can be a single crystalline domain or can have one or more crystalline domains. In one embodiment, the fused nanoparticles are single crystalline. The crystalline phase can be either the thermodynamically favorable crystalline state or a crystalline state which is not thermodynamically favorable but is locked in by the crystallinity of the nanoparticles before fusion. One can vary the thermal treatment in the method of making to achieve a desired crystalline structure, or to covert polycrystalline structures to single crystalline structures.

The length of the nanowire or nanotube can be adapted for use in a transistor device, including use in a channel, and can be, for example, at least about 10 nm in length, at least about 25 nm in length, at least about 50 nm in length, at least about 75 nm in length, or at least about 100 nm in length, or about 250 nm to about 5 microns, or more particularly, about 400 nm to about 1 micron.

The width of the nanowire or nanotube can be adapted for use in a transistor device, including use in a channel, and can be, for example, about 5 nm to about 50 nm, or more particularly, about 10 nm to about 30 nm.

When a plurality of nanowires or nanotubes is present, the lengths and widths can be expressed as average lengths and widths using known statistical methods in materials science. For example, the average length of the nanowire or nanotube can be, for example, about 250 nm to about 5 microns, or more particularly, about 400 nm to about 1 micron. The average width of the nanowire or nanotube can be, for example, about 5 nm to about 50 nm, or more particularly, about 10 nm to about 30 nm.

Also, when a plurality of nanowires or nanotubes is present, the nanowires can be substantially monodisperse in length and/or width. Again, known statistical methods in material science can be used to calculate the polydispersity for length and width. For example, images of the nanowires or nanotubes can be obtained and, for example, 20-50 nanowires can be selected for statistical analysis. The coefficient of variation (CV) can be calculated wherein the standard deviation is divided by the mean. The CV can be, for example, less than about 20%, more preferably, less than about lo %, more preferably, less than about 5%, and more preferably, less than about 3%.

The nanowires or nanotubes can be substantially straight. For example, straightness can be estimated by (1) measuring the true length of the nanowire or nanotube, (2) measuring the actual end to end length, (3) calculate the ratio of true length to actual end to end length. For a perfectly straight nanowire or nanotube, this ratio will be one. In the invention, ratios close to one can be achieved including, for example, less than 1.5, less than 1.2, and less than 1.1.

Inorganic nanowires and nanotubes can be used. Semiconductors are a particularly important type of inorganic nanowire material. The semiconductor material can be, for example, any of the standard types including alloys thereof including IV-IV Group (e.g., Si, Ge, Si₍₁₋ₓ)Geₓ), III-V Group binary (e.g., GaN, GaP), III-V Group ternary (e.g., Ga(As₍₁₋ₓ₎Pₓ)), II-VI Group binary (e.g., ZnS, ZnSe, CdS, CdSe, CdTe), IV-VI Group binary (e.g., PbSe), transition metal oxides (e.g., BiTiO₃), and combinations thereof.

Silicon nanowires, which can be used in the present invention as a preferred embodiment, have been described in the patent literature including, for example, U.S. Pat. Nos. 6,720,240; 6,710,366; 6,707,098; 6,706,402; 6,699,779; 6,643,165; 6,579,742; 6,574,130; 6,515,325; 6,459,095; 6,458,621; 6,432,740; 6,313,015; 6,248,674; 6,103,540; and 5,962,863.

Organic and carbon based nanowires and nanotubes can be used. Carbon nanotubes, which can be used in the present invention, are described in the patent literature in the context of transistor and field effect transistor technology including, for example, U.S. Pat. Nos. 6,689,674; 6,659,598; 6,664,559; 6,590,231; 6,566,704; 6,559,468; 6,515,339; and 6,486,489. They can be single-walled or multi-walled. Carbon nanotube field-effect invertors are described in, for example, Liu et al., Applied Physics Letters, Vol. 79, NO. 20, pages 3329-3331 (Nov. 12, 2001), which is hereby incorporated by reference. See also, Bachtold et al., Science, 294, 1317 (2001). Carbon nanotubes are generally described in, for example, Dresselhaus et al., Science of Fullerenes and Carbon Nanotubes, (Academic, San Diego, Calif., 1996).

Applications and fabrications of nanowires and nanotubes are described in, for example, U.S. patent application publication no. 2003/0089899 (published May 15, 2003) to Lieber et al. and include, for example, field effect transistors, sensors, and logic gates, and this publication is hereby incorporated by reference in its entirety including its description of devices made from nanowires. Additional applications of nanowires are described in, for example, U.S. patent application publication no. 2003/0200521 (published Oct. 23, 2003) to Lieber et al. and include nanoscale crosspoints, which is incorporated by reference in its entirety. Additional applications of nanowires are described in, for example, U.S. patent application publication no. 2002/0130353 (published Sep. 19, 2002) to Lieber et al., and 2002/013311 (also published Sep. 19, 2002) to Lieher et al., and include devices with chemical patterning and bistable devices. Additional applications of nanowires are described in, for example, U.S. patent application publication no. 2002/0117659 (published Aug. 29, 2002) to Lieber et al. and include nanosensors for chemical and biological detection. In addition, applications for related nanorods are described in, for example, U.S. Pat. Nos. 6,190,634; 6,159,742; 6,036,774; 5,997,832; and 5,897,945 to Lieber et al.

To prepare nanowires, in one embodiment, nanocrystals which are linearly disposed and adjacent to each other can be fixed to form nanowires. Fusion can be facilitated by lowering in melting point for nanocrystals resulting from the small dimensions. Examples of semiconducting nanocrystals are known in the art. See, for example, patents and patent publications from Alivisatos including U.S. Pat. Nos. 6,727,065; 6,699,723; 6,440,213; 6,423,551; 6,306,736; 6,225,198; 6,207,392; 5,990,479; 5,751,018; 5,537,000; 5,505,928; and 5,262,357; as well as patent publications from Alivisatos including 2003/0226498; 2003/0145779; 2003/0136943; 2003/0113709; 2003/0100130; 2003/0099968; and 2002/0072234, which are hereby incorporated by reference in their entirety. See also, Peng, Alivisatos et al., Nature, vol. 404, Mar. 2, 2000; 59-61, which is incorporated by reference in its entirety, for synthesis with shape control of CdSe nanocrystals which are termed quantum rods. The structures are quantum confined in at least two of the three axes. The aspect ratio, size, and growth rate of the quantum rods can be systematically controlled by varying the reaction time, the injection and growth temperatures, and the number of injections. Controlling the aspect ratio and shape of nanocrystals is also described in patents to Alivisatos et al., U.S. Pat. Nos. 6,306,736 and 6,225,198, which are hereby incorporated by reference. For group IV nanoparticles and nanocrystals, see also, for example, U.S. Patent applications to Korgel et al. published at 2003/0034486 (Applications of Light Emitting Nanoparticles) and 2003/0003300 (Light Emitting Nanoparticles and Method of Making Same).

If Si channels are used, strained Si can be used.

In particular, nanowires and nanotubes are desired which have surfaces which can be selectively recognized and selectively bound by the biological agent having binding structures. For example, crystalline surfaces and single crystals can facilitate recognition and binding. In addition, precursors to the nanowires and nanotubes can be subjected to recognition and binding by the biological agent. The precursors can be then converted to the nanowire or nanotube. For example, precursor nanocrystals can be used which selectively bind to the biological agent and then the nanocrystals are converted to the nanowire or nanotube.

One aspect of the present invention is how the nanowires or nanotubes are made.

### Chemically Formed Nanowires and Nanotubes

The nanowires and nanotubes can be formed independently of biological moieties including amino acid or nucleic acid-based biological structures such as, for example, peptides, proteins, or viruses. Methods of preparation are described in references noted above including, for example, laser assisted catalytic growth of semiconducting nanowires. For example, nanowires can be prepared without use of biological moieties are described in, for example, in papers from the Lieber group including, for example, Morales et al., Science, vol. 279, 208 (Jan. 9, 1998); Cui et al., J. Phys. Chem. B., 104, 22, Jun. 8, 2000, 5213; Hu et al., Ace. Chem. Res., 32, 435-445 (1999); Duan et al., Adv. Marer., 12, 298-302 (2000); Duan et al., Appl. Phys. Lett., 76, 1116-1168 (2000); Gudiksen et al., J. Am. Chem. Soc., 122, 8801 (2000). See also, Gudiksen et al., Nature, Vol. 415, 617 (Feb. 7, 2002); and Lauhon et al., Nature, Vol. 420, 57 (Nov. 7, 2002), which are hereby incorporated by reference in their entirety. The latter two papers describe heterostructures including superlattices and core-shell structures. These heterostructures can be used to achieve the desired function in the transistor. Moreover, this synthetic strategy can be used to form ends of nanowires which can function as sources and drains.

### Biological Synthesis of Nanowires and Nanotubes and Biological Agents

In addition, the nanowires and nanotubes can be formed with use of biological and organic agents including amino acid or nucleic acid-based biological structures such as, for example, peptides, proteins, or viruses. In particular, nanowires can be made by this route.

Useful methods for the biological synthesis can involve use of scaffolds and can be found in, for example, (i) Mao, Belcher et al., Science, 303, 213-217, Jan. 9, 2004, (ii) U.S. provisional patent application to Belcher, Mao, and Solis, Ser. No. 60/534,102 filed Jan. 5, 2004 (“Inorganic Nanowires”), (iii) “Biological Control of Nanoparticle Nucleation, Shape, and Crystal Phase;” 2003/0068900 published Apr. 10, 2003; and (iv) “Biological Control of Nanoparticles;” 2003/0113714 published Jun. 19, 2003; which are each incorporated by reference in their entirety.

More particularly, the present invention provides, in one embodiment, an inorganic nanowire having an organic scaffold substantially removed from the inorganic nanowire, the inorganic nanowire consisting essentially of fused inorganic nanoparticles substantially free of the organic scaffold. The present invention also can be practiced with compositions comprising a plurality of these inorganic nanowires to prepare a plurality of devices including integrated devices. This invention also provides compositions comprising a plurality of inorganic nanowires, wherein the inorganic nanowires comprise fused inorganic nanoparticles substantially free of organic scaffold.

The organic scaffold is generally removed so that, preferably, it cannot be detected on the nanowire. This substantial removal can be described in terms of weight percentage remaining. For example, the amount of remaining organic scaffold with respect to the total amount of nanowire and scaffold can be less than 1 wt. %, more preferably, less than 0.5 wt. %, and more preferably, less than 0.1 wt. %. A basic and novel feature of this embodiment of the invention is the substantial removal of the scaffold in the production of high quality nanowires.

In another patent application, which is hereby incorporated by reference in its entirety, [U.S. Ser. No. 10/665,721 filed Sep. 22, 2003 to Belcher et al. (“Peptide Mediated Synthesis of Metallic and Magnetic Materials”)], additional description is provided for burning off and elimination of a viral scaffold from materials to which the scaffold can selectively bind. In this application, annealing temperatures of 500-1,000° C. are described for burning off the scaffold.

### Scaffold and Binding

Although the scaffold ultimately can be substantially removed from the nanowire, the scaffold is an important part of this embodiment of the invention. Moreover, the technology described in the above-noted references and further described below for the scaffold can also be adapted for use in designing the biologic agent comprising binding structures, which is also described further below. The biological agent comprising binding structures can be adapted to bind nanoparticles and nanocrystals, as well as nucleate and catalyze synthesis of nanoparticles and nanocrystals, which are of use in fabrication of transistor elements including channels, dielectrics, gates, sources, and drains.

In the practice of the present invention, one skilled in the art can refer to technical literature for guidance on how to design and synthesize the scaffold including the literature cited herein. Although the present invention relates to organic scaffolds and is not limited only to viral scaffolds in its broadest scope, viral scaffolds are a preferred embodiment. In particular, an elongated organic scaffold can be used which is a virus, and the term virus can include both a full virus and a virus subunit such as a capsid. The literature describes the preparation of viral scaffolds through genetic engineering with recognition properties for exploitation in materials synthesis. This includes use of viruses in the production of inorganic materials which have technologically useful properties and nanoscopic dimensions. In the present invention, one skilled in the art can use the literature in the practice of the present invention to prepare inorganic nanowires on scaffolds, wherein the scaffolds are later substantially eliminated so that the inorganic nanowire is substantially free of the scaffold.

One skilled in the art, for example, can refer to the following patent literature for selection of the virus, genetic engineering methods, and for materials to be used with genetically engineered viruses. Phage display libraries and experimental methods for using them in biopanning are further described, for example, in the following U.S. patent publications to Belcher et al.: (1) “Biological Control of Nanoparticle Nucleation, Shape, and Crystal Phase;” 2003/0068900 published Apr. 10, 2003; (2) “Nanoscale Ordering of Hybrid Materials Using Genetically Engineered Mesoscale Virus;” 2003/0073104 published Apr. 17, 2003; (3) “Biological Control of Nanoparticles;” 2003/0113714 published Jun. 19, 2003; and (4) “Molecular Recognition of Materials;” 2003/0148380 published Aug. 7, 2003, which are each hereby incorporated by reference in their entirety. Additional patent applications useful for one skilled in the art describe viral and peptide recognition studies with use of genetically engineered viruses for materials synthesis and applications including, for example, (1) U.S. Ser. No. 10/654,623 filed Sep. 4, 2003 to Belcher et al. (“Compositions, Methods, and Use of Bi-Functional BioMaterials”), (2) U.S. Ser. No. 10/665,721 filed Sep. 22, 2003 to Belcher et al. (“Peptide Mediated Synthesis of Metallic and Magnetic Materials”), and (3) U.S. Ser. No. 10/668,600 filed Sep. 24, 2003 to Belcher et al. (“Fabricated BioFilm Storage Device”), (4) U.S. provisional Ser. No. 60/510,862 filed Oct. 15, 2003 to Belcher et al. (“Viral Fibers”), and (5) U.S. provisional Ser. No. 60/511,102 filed Oct. 15, 2003 to Belcher et al. (“Multifunctional Biomaterials . . . ”); each of which are hereby incorporated by reference. These references describe a variety of specific binding modifications which can be carried out for binding to conjugate structures, as well as forming the conjugate structures in the presence of the material modified for specific binding. In particular, polypeptide and amino acid oligomeric sequences can be expressed on the surfaces of viral particles, including both at the ends and along the length of the elongated virus particle such as M13 bacteriophage, including pIII and pVIII expressions, as well as PIX, pVII, and pVI expressions, and combinations thereof. A single site for modification can be modified with more than one unit for specific binding. For example, a pVIII site can be modified to have two distinctly different binding units. The scaffold can be functionalized with sufficient binding units to achieve the desired concentration needed to form the nanowire.

One skilled in the art can also refer to, for example, C. E. Flynn et al. Acta Materialia, vol. 13, 2413-2421 (2003) entitled “Viruses as vehicles for growth, organization, and assembly of materials.” This reference, as well as all references cited in the specification, are incorporated herein by reference in their entirety. For example, section 2 of this paper, and references cited therein, describe peptide selection of specific material recognition motifs; section 3 describes controlled nucleation and growth of inorganic materials; section 4 describes use of viruses as nanowire templates; and section 5 describes self-assembly of nanomaterials into liquid crystals, films, and fibers using genetically engineered viruses. In addition, the reference (Mao et al., Proc. Natl. Acad Sci, 2003, 100, 6946) is hereby incorporated by reference for all of its teachings including the nucleation and structures shown in FIG. 1. Also, in particular, the reference (Flynn et al., J. Mater. Chem, 2003, 13, 2414-2421) is also incorporated by reference in its entirety including descriptions of using aqueous salt compositions to nucleate nanocrystals which are directed in their crystal structure and orientation by the recognition sites. In the present invention, these nucleated nanocrystals can be converted to single crystalline and polycrystalline nanowires, wherein the scaffold is substantially removed. See also, Reese, Belcher et al. Nanoletters, (“Biological Routes to Metal Alloy Ferromagnetic Nanostructures”), 2004, which is incorporated by reference in its entirety.

The scaffold is further described including the role of genetic programming for the preferred embodiments. Although the viral scaffolds represent a preferred embodiment, the present invention comprises other types of non-viral scaffolds as well. Also, although M13 virus is a preferred embodiment for a scaffold, the present invention is not limited to this virus.

### Peptide/Virus Embodiment

The scaffold, which can control nanowire, nanotube, and transistor fabrication in the present invention, can comprise an entire virus, a virion, or viral subunits including capsids. Viral subunits including proteins, peptides, nucleic acids, DNA, RNA, and the like, in various combinations. The scaffold does not require that both peptide and nucleic acid be present. For example, virus mimics can be used or engineered, wherein the size, shape, or structure mimics that of a virus, but the does not contain nucleic acid and/or may not have the ability to infect a host for replication. One skilled in the art can prepare viral scaffolds based on purely synthetic or engineering methods from the bottom up as well as using more traditional methods wherein materials are supplied by nature without or without modification by man.

In a preferred embodiment, wherein the scaffold is a virus or a virus subunit, the scaffold is tailored and designed in structure and function by genetic programming and/or genetic engineering for production of the one dimensional materials such as nanowires. The genetic programming can be used to tailor the scaffold for the particular application, and applications are described further below. See, e.g., Genetically Engineered Viruses, Christopher Ring and E. D. Blair (Eds.), Bios Scientific, 2001, for descriptions of developments and applications in use of viruses as vehicles and expressors of genetic material including, for example, prokaryotic viruses, insect viruses, plant viruses, animal DNA viruses, and animal RNA viruses. In the present embodiment of the invention, genetic programming can be carried out to engineer a scaffold using the different displayed peptide features of a virus such as, for example, a filamentous bacteriophage such as, for example, the M13 virus which has a rod shape. Genetic programming can be used to control the scaffold for materials synthesis, the viral scaffold comprising one or more viral particle subunits which may or may not include the nucleic acid subunit of the virus. Also, the scaffold may or may not retain infectability.

An overall commercial advantage to this genetic programming approach to materials engineering, in addition to materials-specific addressability, is the potential to specify viral length and geometry, and hence nanowire or nanotube length and geometry. Hence, a variety of methods can be used to control the scaffold length and geometry.

For example, the length of a filamentous virus is generally related to the size of its packaged genetic information and the electrostatic balance between the pVIII-derived core of the virion and the DNA. [See, e.g., B. K. Kay, J. Winter, J. McCafferty, Phage Display of Peptides and Proteins: A Laboratory Manual, Academic Press, San Diego, 1996.] Phage observed by AFM generally are seen to be roughly 860 nm and as short as 560 nm depending on whether the complete M13 genome or smaller phagemid are used in sample preparation. [See, e.g., C. Mao, C. E. Flynn, A. Hayhurst, R. Sweeney, J. Qi, J. Williams, G. Georgiou, B. Iverson, A. M. Belcher, Proc. Natl. Acad. Sci. 2003, 100, 6946.] Also, changing a single lysine to glutamine on the inner-end of pVIII can result in particles approximately 35% longer than wild type phage. [See, e.g., J. Greenwood, G. J. Hunter, R. N. Perham, J. Mol. Biol. 1991, 217, 223.]

In addition, specific linkage, binding, and concatenation of virus particles can help produce longer viral scaffolds, and thus longer nanowires. The multiplicity of additions can be controlled by engineering binding motifs into one virus, which then can accurately recognize binding sites on another virus. For example, the pIII protein resides at one end of the M13 virus and can be exploited to display peptide and protein fusions. At the other end of the virus, the pIX and pVII proteins also can be subject to modification. For example, Gao and coworkers utilized pIX and pVII fusions to display antibody heavy- and light-chain variable regions. [See, e.g., C. Gao, S. Mao, G. Kaufmann, P. Wirsching, R. A. Lerner, K. D. Janda, Proc. Nail. Acad. Sci. 2002, 99, 12612.] See, also, for example, U.S. Pat. No. 6,472,147 for genetic modification of viruses. This invention encompasses dual-end viral display, either for generating bimodal heterostructures, or in combination with pVIII, producing end-functionalized nanowires.

In addition, dual-end directional linkages enable creation of other interesting and commercially useful geometries, such as rings, squares and other arrays. The binding of one end of a virus directly to the other end of the virus without the use of a linker can be used to form rings, wires, or other viral based structures as well. By engineering recognition sites and the corresponding conjugate moieties into a single virus, or multiple viruses, the entire system can be genetically programmed.

When a nanowire can be represented by [X-Y]n wherein n represents repeating units of different compositions X and Y, different viral units can provide the X and Y component. Similarly, when nanowires can also be represented by S-C-D structures wherein S is the source, C is the channel, and D is the drain, different viral units can provide the S, C, and D structures.

An important advantage of the invention is that the organic scaffold can be an active scaffold, wherein the scaffold not only serves as a template for synthesis of the inorganic nanowire, but also actively assists in coupling the inorganic nanowire to other structures. For example, an organic scaffold which is designed at one end to bind to another structure can be used to couple the inorganic nanowire to the structure. The scaffolds and the nanowires can be coupled to each other, for example, to form segments of similar or dissimilar materials. In this embodiment, the composition of the nanowire would vary as a function of length. Additional description is provided for the types of viral structures which can be designed by genetic programming for particular applications based on length control, geometry control, binding control, and the like. The virus scaffold is not particularly limited, and combinations of viruses can be used of different types. In general, viruses can be used which can be multifunctionalized. In general, virus particles which are long, filamentous structures can be used. See, e.g., Genetically Engineered Viruses, Christopher Ring (Ed.), Bios Scientific, 2001, pages 11-21. Additionally, other viral geometries such as dodecahedral and icosahedral can be multifunctionalized and used to create composite materials. Virus particles which can function as flexible rods, forming liquid crystalline and otherwise aligned structures, can be used.

In particular, phage display libraries, directed evolution, and biopanning are an important part of genetic programming of viruses, and viruses can be used which have been subjected to biopanning in the viral design so that the virus particles specifically can recognize and bind to materials which were the object of the biopanning. The materials can also be nucleated and synthesized in particulate form, including nanoparticulate form, in the presence of the specific recognition and binding sites. Use of filamentous virus in so called directed evolution or biopanning is further described in the patent literature including, for example, U.S. Pat. Nos. 5,223,409 and 5,571,698 to Ladner et al. (“Directed Evolution of Novel Binding Proteins”). Additional references on the recognition properties of viruses include U.S. Pat. No. 5,403,484 (phage display libraries, now commercially available) and WO 03/078451.

Mixtures of two or more different kinds of viruses can be used. Mixtures of virus particles with non-virus materials can be used in forming materials which use the present invention.

Virus and virus particle can include both an entire virus and portions of a virus including at least the virus capsid. The term virus can refer to both viruses and phages. Entire viruses can include a nucleic acid genome, a capsid, and may optionally include an envelope. Viruses as described in the present invention may further include both native and heterologous amino acid oligomers, such as cell adhesion factors. The nucleic acid genome may be either a native genome or an engineered genome. A virus particle further includes portions of viruses comprising at least the capsid.

In general, a virus particle has a native structure, wherein the peptide and nucleic acid portions of the virus are arranged in particular geometries, which are sought to be preserved when it is incorporated in solid state, self supporting forms such as films and fibers.

For transistor fabrication, viruses are preferred which have expressed peptides, including peptide oligomers and amino acid oligomer as specific binding sites. Amino acid oligomers can include any sequence of amino acids whether native to a virus or heterologous. Amino acid oligomers may be any length and may include non-amino acid components. Oligomers having about 5 to about 100, and more particularly, about 5 to about 30 amino acid units as specific binding site can be used. Non-amino acid components include, but are not limited to sugars, lipids, or inorganic molecules.

The size and dimensions of the virus particle can be such that the particle is anisotropic and elongated. Generally, the viruses may be characterized by an aspect ratio of at least 25, at least 50, at least 75, at least 100, or even at least 250 or 500 (length to width, e.g, 25:1).

A wide variety of viruses may be used to practice the present invention for transistor fabrication. The compositions and materials of the invention may comprise a plurality of viruses of a single type or a plurality of different types of viruses. Preferably, the virus particles comprising the present invention are helical viruses. Examples of helical viruses include, but are not limited to, tobacco mosaic virus (TMV), phage pf1, phage fd1, CTX phage, and phage M13. These viruses are generally rod-shaped and may be rigid or flexible. One of skill in the art may select viruses depending on the intended use and properties of the virus.

Preferably, the viruses of the present invention have been engineered to express one or more peptide sequences including amino acid oligomers on the surface of the viruses. The amino acid oligomers may be native to the virus or heterologous sequences derived from other organisms or engineered to meet specific needs.

A number of references teach the engineering of viruses to express amino acid oligomers and may be used to assist in practicing the present invention for transistor fabrication. For example, U.S. Pat. No. 5,403,484 by Ladner et al. discloses the selection and expression of heterologous binding domains on the surface of viruses. U.S. Pat. No. 5,766,905 by Studier et al. discloses a display vector comprising DNA encoding at least a portion of capsid protein followed by a cloning site for insertion of a foreign DNA sequence. The compositions described are useful in producing a virus displaying a protein or peptide of interest. U.S. Pat. No. 5,885,808 by Spooner et al. discloses an adenovirus and method of modifying an adenovirus with a modified cell-binding moiety. U.S. Pat. No. 6,261,554 by Valerio et al. shows an engineered gene delivery vehicle comprising a gene of interest and a viral capsid or envelope carrying a member of a specific binding pair. U.S. Published Patent Application 2001/0019820 by Li shows viruses engineered to express ligands on their surfaces for the detection of molecules, such as polypeptides, cells, receptors, and channel proteins.

For transistor fabrication in the present invention, M13 systems are a preferred example of a filamentous virus scaffold. The wild type filamentous M13 virus is approximately 6.5 nm in diameter and 880 nm in length. The length of the cylinder reflects the length of the packaged single stranded DNA genome size. At one end of M13 virus, there are approximately five molecules each of protein VII (pVII) and protein IX (PIX). The other end has about five molecules each of protein III (pIII) and protein VI (pVI), totaling 10-16 nm in length. The wild type M13 virus coat is composed of roughly 2800 copies of the major coat protein VIII (pVIII) stacked in units of 5 in a helical array.

In sum, evolution of substrate specific peptides through phage display technologies for the directed nucleation of materials on the nanometer scale has been previously reported by papers and patents from Angela Belcher and coworkers and serves as the basis for the material specificity in the virus scaffold or template of the present invention. Screening phage libraries for the ability to nucleate and assemble inorganic systems including, for example, the ZnS, CdS, FePt and CoPt systems using commercially available bacteriophage libraries expressing either a disulphide constrained heptapeptide or a linear dodecapeptide, has yielded consensus sequences. Incorporation of these peptides into the highly ordered, self assembled capsid of the M13 bacteriophage virus provides a linear template which can simultaneously control particle phase and composition, while maintaining an ease of material adaptability through genetic tuning of the basic protein building blocks. Because the protein sequences responsible for the materials growth are gene linked and contained within the capsid of the virus, exact genetic copies of this scaffold are relatively easily reproduced by infection into a large suspension of bacterial hosts.

To prepare nanowires, an anisotropic scaffold can be used which has the ability to collect nanoparticles being formed around it and locate them on the scaffold for fusion into a nanowire. In this invention, an inorganic nanowire composition can be formed having a scaffold substantially removed from the inorganic nanowire. Non-viral scaffolds can also be used including, for example, a variety of other organic scaffolds including, for example, scaffolds which have peptide or protein recognition units as side groups on an organic backbone. For example, the organic backbone can be a synthetic polymer backbone as well known in the art. For example, polymer scaffolds can be used including for example modified polystyrenes of uniform molecular weight distribution which are functionalized with peptide units. Another example is branched polypeptides or nucleic acids which are modified to have recognition sites. Another example is a nanolithographically printed peptide structures such as a line with nanoscale width. In general, DNA, proteins, and polypeptides can be modified with recognition units, including peptide recognition units, to function as the organic scaffold.

In one embodiment, scaffolds and virus particles can be used which are not directly genetically engineered. However, in general, desirable properties can be achieved when the virus is genetically engineered or genetic engineering is used in designing the scaffold.

Scaffolds can be surface coated with nanocrystals and the coating can be carried out on an exterior surface or an interior surface. For example, some viruses and virus capsids can have internal opening with internal surfaces. By genetic engineering, recognition and binding structures can be introduced into interior surfaces as well as exterior surfaces. Hence, nanocrystalline growth and binding can occur on the interior surface as well. Interior surface can be in the form of channels or cages. See, e.g., Douglas et al., Adv. Mater., 2002, 14, 415; Douglas et al., Adv. Mater., 1999, 11, 679; and Douglas et al., Nature, 1998, 393, 152. See, also, Shenton, Douglas, Mann et al., Adv. Mater., 1999, 11, 253 including description of a 4 nm wide interior cavity for TMV and particle growth therein.

The nanowires and nanotubes can function as a channel and can be combined with dielectric materials to separate the channel from the gate.

### Dielectric Materials and High K Dielectric Layers

Both standard K and high K dielectric materials can be used and are known in the art including oxides and metal oxides, although high K dielectrics are preferred. For example, standard-K dielectric materials generally have a K up to about 10. Such standard-K dielectric materials include, for example, silicon dioxide, which has a K of about 4, silicon oxynitride, which has a K of about 4-8 depending on the relative content of oxygen and nitrogen, silicon nitride, which has a K of about 6-9, and aluminum oxide, which has a K of about 10. High-K dielectric materials can have generally a K greater than about 10. Such high-K dielectric materials include, for example, HfO₂, ZrO₂, TiO₂, and others known in the art, some of which are specifically identified more fully below. Dielectrics can have a K value of about 1 to about 4 in one embodiment, and about 4 to about 10 in another embodiment. In general, high-K dielectric materials encompass binary, ternary and higher dielectric oxides and ferroelectric materials having a K of about 10 or more. High-K dielectric materials may also include, for example, composite dielectric materials such as hafnium silicate, which has a K of about 14, and hafnium silicon nitride, which has a K of about 18. Hafnium-based and zirconium-based compositions are preferred. The dielectric should provide good insulating properties and create high capacitance between gate and channel. It should allow thicker dielectric layers to be used which prevents undesirable leakage between gate and channel.

The preferred high-K dielectric material can comprise at least one of hafnium oxide(HfO₂), zirconium oxide(ZrO₂), tantalum oxide(Ta₂O₅), barium titanate (BaTiO₃), titanium dioxide(TiO₂), cerium oxide(CeO₂), lanthanum oxide(La₂O₃), lead titanate (PbTiO₃), silicon titanate (SrTiO₃), lead zirconate (PbZrO₃), tungsten oxide (WO₃), yttrium oxide (YO₃), bismuth silicon oxide (BiSi₂O₁₂), barium strontium titanate (BST)(Ba₁₋ₓSrₓTiO₃), PMN (PbMgₓNb₁₋ₓO₃), PZT(pbZrₓTi₁₋ₓO₃), PZN (PbZnₓNb₁₋ₓO₃), and PST(PbScₓTa₁₋ₓO₃). Nitrided hafnium silicates can be used.

The patent literature describes high K dielectric layers, materials, and various uses in microelectronic devices including, for example, U.S. Pat. Nos. 6,730,576; 6,706,581; 6,682,973; 6,673,669; 6,657,267; 6,656,852; 6,656,764; 6,638,876; 6,630,712; 6,620,713; 6,599,766; 6,596,596; 6,580,115; 6,566,205; 6,563,183; 6,555,473; 6,514,829; 6,509,234; 6,495,437; 6,475,856; 6,455,424; 6,452,229; 6,451,641; 6,444,592; 6,391,801; 6,380,104; 6,380,038; 6,351,005; 6,348,385; 6,320,238; 6,271,084; 6,218,693; 6,194,748; 6,165,834; 6,124,164; and 6,020,024, all of which are incorporated by reference in their entirety.

The biological agent comprising binding structures can bind to the dielectric in a variety of forms including a nanoparticle form, a nanocrystal form, patterned form, surface crystalline form, a precursor form, and other forms. Hence, the dielectric can be disposed close to the channel and contact the channel. Removal of the biological agent can be carried out by heat treatment. This can improve the interfacial characteristics of the dielectric and channel. The thickness of the dielectric layer can be controlled by the biological agent binding and control of particle size for example. The dielectric also can be in proximity and contact with the gate layer.

The dielectric can fully surround the channel, and the gate can fully surround the dielectric.

### Gate Materials and Layers

Gate layer materials are known in the art including gate silicon and gate metal materials. See, for example, U.S. Pat. No. 6,638,824. If desired, the gate metal layer can be formed by a variety of processes, including physical vapor deposition (sputtering), evaporation, chemical vapor deposition, plating, or a combination of these or other methods. It can also be formed using the biological agent. Typical metals include aluminum-silicon-copper alloy, tungsten, tungsten over titanium nitride, tantalum nitride, gold over titanium nitride, titanium/gold, platinum, and copper, although other metals or combination of metals or metals and barrier/adhesion layers may be used. Such metals are compatible with semiconductor processes and have a lower resistivity than polysilicon when used as a gate conductor. Metals can be used to avoid undesirable threshold voltage pinning. Metal selection can be varied depending on whether PMOS or NMOS applications are at hand.

The gate can be part of a wrap around gate structure. The gate can be a metallic gate. The gate can have a gate length of about 100 nm or less. As gate lengths become smaller, gate length can be 90, 80, 70, 60, 50, 40, 30, 20, and 10 nm or less.

The channel also can be in electrical communication with the source and drain, although the source and drain need to be decoupled from the gate.

Metals can be deposited onto the ends of the nanowires and nanotubes including, for example, gold to form gold metal electrodes.

Both high-K/metal gate transistors and trigate transistors can be the subject of the present invention as they are each candidates for 45 nm transistors and terahertz transistors.

### Sources and Drains

Known transistor source and drain materials can be used, and references described above with respect to the gates, dielectrics, and channels further describe potential sources and drains. Geometries can be planar or elevated. Nanolithography and other patterning methods can be used to prepare them. The source can be the part of the transistor where current flows from, and the drain can be the part of the transistor where current flows to. Both source and drain can be, for example, doped silicon. The source and drain can be crystalline in surface structure to facilitate binding by a biological agent. The transistor can be symmetrical wherein current can flow from source to drain, or alternatively adapted to flow from drain to source. The source and drain can be nanostructures and can have length and width dimensions which are about 500 nm or less, or have a length or width of about 100 nm or less. The sizes can be adapted to follow the international standards noted above by node. Geometry, size, and material selection can be adapted for large scale integration on a substrate. In general, ultra-shallow source and drain structures are desired which can be produced by, for example, ion-implantation. Very low energy ion beams can be used in combination with reduced thermal budget. Technologies which can be used in, for example, the 65 nm node devices include, for example, uniform/HALO doping, super steep retrograde/steep HALO doping, ultra low energy implant, plasma doping, and selective epi.

For embodiments wherein the channel comprises a nanotube or a nanowire, sources and drains can be (i) part of the nanotube or nanowire channel as part of an integrated structure (e.g., a modulated nanowire structure with source and drain at ends and channel in the center), (ii) distinct from but contacting the nanotube or nanowire in a good interfacial relationship which provides functional contact, or (iii) both (i) and (ii).

### Combination of Transistor Elements with Biological Agents

Biological agents are in part described above with respect to use of scaffolds and binding, including use of peptides and viruses, to form nanowires and nanotubes. The biological agent is not particularly limited as long as it comprises at least two binding structures and can assist in the fabrication of the transistor and its elements. The methods described above for use of biological agents to form nanowires and nanotubes can be also used to identify binding structures for the materials and elements of the present invention which are to be combined by the biological agent. Each of the binding structures can bind to one of the elements. For example, one binding structure can bind to the dielectric and one binding structure can bind to the channel. Alternatively, one binding structure can bind to the dielectric and one binding structure can bind to the gate. The biological agent can comprise nucleic acid, whether of RNA or DNA types, or amino acids, whether low molecular or high molecular types. Aptamer binding can be utilized. Lipid binding can be utilized. Combinations of natural and synthetic systems can be used. The biological agent can be for example a synthetic or engineered peptide. The biological agent can be a peptide comprising peptide binding structures. The biological agent can be, for example, a multifunctional peptide and in particular a bifunctional peptide. The biological agent can be introduced into the fabrication system to perform its fabrication function by combining parts and then it can be removed as desired by controlled heating, annealing, and thermal degradation and vaporization.

Electrical devices which comprise biological components, including use of binding structures, are described in for example U.S. Pat. No. 6,703,660 to Yitzchaik et al. See also, K. Keren et al., Science, 302, 1380-1382, Nov. 21, 2003 (“DNA Templated Carbon Nanotube Field Effect Transistor”).

A preferred embodiment is a bifunctional biological agent which can be represented by the generic structure A-B-C, wherein B is an optional spacer between binding moieties A and C. A and C can comprise, for example, peptide, nucleic acid, and lipid structures which can provide selective binding to transistor components and precursors thereof.

A preferred embodiment is a bifunctional peptide, or peptide linker, which can be represented by the generic structure A-B-C, wherein B is an optional spacer between peptide binding moieties A and C. Binding moieties A and B for example can be identified by other methods and then synthesized in combination with each other and the optional B moiety. In one embodiment, all units in A, B, and C are peptide units. Binding structures can be identified by, for example, phage display and directed evolution, or by mixing combinatorial mixtures with preexisting nanoparticles or nanocrystals, and separating bound from unbound structures from the initial combinatorial set. Examples of bifunctional peptides in the patent literature include, for example, U.S. Pat. Nos. 6,086,881; 6,420,120; and 6,468,731.

Bi-functional binding and multifunctional binding are described in, for example, (i) U.S. Ser. No. 10/654,623 filed Sep. 4, 2003 to Belcher et al. (“Compositions, Methods, and Use of Bi-Functional BioMaterials”); and (ii) U.S. provisional Ser. No. 60/511,102 filed Oct. 15, 2003 to Belcher et al. (“Multifunctional Biomaterials . . . ”), which are incorporated by reference in their entirety.

One embodiment is an assembly embodiment. In one embodiment, the material of interest such as, for example, a channel material, a dielectric material, a gate material, a source material, or a drain material, is provided in form which the biological agent can selectively recognize and bind to. For example, the form can be a nanoparticle or a nanocrystal. It can be a single crystal. The biological agent can be used to assemble nanoparticles and nanocrystals wherein the biological agent controls the location of the bound material with respect to other bound material as well as unbound material.

In another embodiment, the material of interest such as channel, dielectric, gate, source, or drain material is synthesized in the presence of the biological agent, wherein the biological agent controls the synthesis of the material. For example, the biological agent can catalyze or nucleate material of interest. In many cases, it will also bind to the material as it forms.

In addition to peptide technology, aptamer technology can be used. Methods for making and modifying aptamers, and assaying the binding of an aptamer to a target molecule may be assayed or screened for by any mechanism known to those of skill in the art (see for example, U.S. Pat. Nos. 6,111,095, 5,861,501, 5,840,867, 5,792,613, 5,780,610, 5,780,449, 5,756,291, 5,631,146 and 5,582,981; as well as PCT Publication Nos. WO92/14843, WO91/19813, and WO92/05285, each of which is incorporated herein by reference). Aptamers are single- or double-stranded DNA or single-stranded RNA molecules that recognize and bind to a desired target molecule by virtue of their shapes. See, e.g., PCT Publication Nos. WO92/14843, WO91/19813, and WO92/05285. The SELEX procedure, described in U.S. Pat. No. 5,270,163 to Gold et al., Tuerk et al. (1990) Science 249:505-510, Szostak et al. (1990) Nature 346:818-822 and Joyce (1989) Gene 8233-87, can be used to select for RNA or DNA aptamers that are target-specific. In the SELEX procedure, an oligonucleotide is constructed wherein an n-mer, preferably a random sequence tract of nucleotides thereby forming a “randomer pool” of oligonucleotides, is flanked by two polymerase chain reaction (PCR) primers. The construct is then contacted with a target molecule under conditions which favor binding of the oligonucleotides to the target molecule. Those oligonucleotides which bind the target molecule are: (a) separated from those oligonucleotides which do not bind the target molecule using conventional methods such as filtration, centrifugation, chromatography, or the like; (b) dissociated from the target molecule; and (c) amplified using conventional PCR technology to form a ligand-enriched pool of oligonucleotides. Further rounds of binding, separation, dissociation and amplification are performed until an aptamer with the desired binding affinity, specificity or both is achieved. The final aptamer sequence identified can then be prepared chemically or by in vitro transcription.

The bifunctional biological agent, which can be described by the generic formula A-B-C, may comprise binding moieties, A and C, with different levels of materials specificity depending on the application or desired result. For example, if the biological agent is used to create a gate region by binding to a metal material (e.g., Cu) through moiety A on a cylindrical dielectric region bound through C (e.g., HfO₂), then A can comprise a generic metal binding or nucleation agent such as a thiol containing molecule (e.g., which binds to Cu), whereas C can be a very specific binding structure that directs metal formation only on the dielectric surface.

### Selective Attachment into Circuit and Parallel Fabrication

As noted above, the biological agent comprising binding structures can direct transistor elements to desired locations, and this can also be used to bind and direct transistors and transistor elements to larger substrates and circuits, so that integrated circuits become possible. Transistors can be combined into logic gates and memory components as known in the art to generate computing power.

For example, a surface can be patterned with compositions on the surface, and the biological agent can deliver the element of interest to the composition on the surface. This can result in parallel fabrication of thousand and millions of binding events happening at once in an organized spatial pattern onto a single surface. Patterning can be carried out by known nanolithographic methods.

Hence, the present invention further comprises a patterned surface and one or more biological agents selectively bound to the patterned surface and which function to link the patterned surface to one or more transistor elements of interest.

Self assembly can be used to generate patterned structures in parallel fabrication. For example, a patterned substrate can provides and then transistor components can self assemble on top of the patterned areas, providing an integrated circuit of preset design.

Wafer size is not particularly limited but wafer sizes of 200 and 300 mm can be used to prepare larger device structures.

### Engineering the Surface of Nanowire and Nanotube with Outer Layer

Although the particular focus of this application is with nanotransistor fabrication, the invention comprises compositions and methods which can be used for other applications as well. For example, the nanowire or nanotube can be engineered to have a first outer layer, and then also a second outer layer, each outer layer cocentric with the core nanowire or nanotube. The biological agent can be used to bind the core nanowire or the nanotube with the first outer layer, or the first outer layer to the second outer layer. The first outer layer can be a dielectric material including a high-K dielectric material as described above. The second outer layer can be a conductive material including a gate material such as poly-silicon or metal.

### Preferred Embodiments

To build a wrap-around gate according to the present invention, looking at FIG. 2, three stages can be carried out, one stage for the creation of each layer. For example, stage 1 can be the formation of the Si nanowire core. Stage 2 can be the formation of the dielectric layer around the core Si wire. And stage 3 can be the formation of the gate material layer around the dielectric. Two approaches can be adopted depending on whether the nanowire is chemically fabricated or biologically synthesized.

Approach 1: Nanowire FET Starting from Chemically Fabricated Semiconductor nanowires

Stage 1: Chemical Formation of Heterogenous Nanowires:

Si nanowires are obtained from an outside source or are prepared using known published methods to create Si nanowires. The wires are obtained or formulated through chemical processes. For instance, wires are made by methods reported from Charlie Lieber's Group (e.g., FIG. 6, Science 279, 208, 1998).

The ends of the wires can be made distinguishable from the body of the wire so that they may act as or couple to source and drain regions. For example, the nanowires can be made as free standing channel regions surrounded by dielectric and gate regions that are directed to, bound to, and fused with source and drain regions on a separate circuit via bifunctional agents; or they can have source and drain regions grown or assembled on the ends of the nanowire before incorporation into the circuit. A third alternative would involve growing or assembling small regions of source and drain material on the nanowire ends and binding these ends to larger source and drain regions on an existing circuit element via bifunctional agents and subsequently fusing these materials.

There are known ways to modify each end of a carbon nanotube/Si wire, and these methods can be used to make each end unique. In one example (Leiber et al. Nature, 415, 617-620, 2002) silicon nanowires are grown from a gold “seed” catalysts in the presence of precursor gases. Changing the gas composition changes the dopant level of the growing crystal nanowire, thus creating n-doped and p-doped ends of the nanowire. Alternatively, the silicon crystal composition can be modified at the nanowire ends in such a way that it can be recognized by and linked with a bifunctional peptide to a doped material, thus coupling the Channel region (nanowire) to the source and drain regions.

Stage 2: Formation of the Dielectric Layer (Assemble or Nucleate)

There can be two choices for this, and the choice is mainly dependant on the type of material to be the high K dielectric layer.

If one chooses SiO2, assuming that trapped charges, leakage and breakdown characteristics of SiO2 are acceptable in this new device geometry, then the Si wire that was synthesized through nonbiological processes can easily be oxidized to form a layer of SiO2 (Lieber Science Vol279, Pg218).

If one chooses a different dielectric material (or one wants a more controlled deposition of SiO2), then the dielectric layer can be assembled and/or nucleated by specific bifunctional peptides. On one end can be a peptide that nucleates or binds to a dielectric nanoparticle with controlled size, while on the other end can be a peptide that would bind to the Si nanowire. At this point, to create the optimal interface, the dielectric layer formation can be followed with an annealing step to ensure a good interface between the dielectric and the Si wire. On the other hand, annealing may be carried out after another deposition step or after final device assembly.

Stage 3: Formation of Gate Layer (Assemble or Nucleate)

To assemble/nucleate the third layer, the gate material can be a metal. One can use another bifunctional peptide. One end of the peptide can nucleate or bind the gate material, controlling its thickness by nanoparticle size, while the other end would bind to the dielectric layer. Again at this stage, to create the optimal interface, gate layer formation can be followed with an annealing step to ensure a good interface between the layers.

In carrying out these 3 stages, the modified ends of the nanowires can be preserved throughout each step.

Approach 2: Nanowire FET from Biologically Templated Semiconductor Nanowires

Stage 1: Biological Synthesis of the Core Si Nanowire:

To create the core Si nanowire, one can use viruses as templates to form the Si nanowire. The virus can be engineered to either grow or assemble nanocrystals forming a wire inside or outside the viral capsid coat (see, for example, description of Tobacco Mosaic Virus by Shenton, Douglas, Mann et al., Adv. Muter., 1999, 11, 253 including a 4 nm wide interior cavity, and M13 by as described in Belcher, Acta Materialiu review article cited above and Nanoletters, 2003, vol 3, no. 3,413). The ends of the virus (p3 and p719 in M13 bacteriophage) can be uniquely engineered with peptides to bind to or grow a source and a drain region at opposite ends (doped n or p type semiconductor material). These source and drain material ends can then be coupled to an existing circuit element via bifunctional peptides as described in approach 1, stage 1. As long as the viral template remains, this end modification may take place either in Stage 1 or Stage 2. Alternatively, the viral ends may contain peptides that directly bind to the source and drain regions on an existing circuit element, thus eliminating the need for a free-standing bifunctional agent to locate the nanowire into the circuit.

If one templates the wire on the outside of the viral capsid, we may anneal at this point. If we template the wire on the inside of the viral capsid, annealing may wait, especially if the outside of the viral template is exploited as a binding/nucleation element in the next stage.

Stage 2: Template/Bifunctional Peptide Dielectric (Assemble or Nucleate):

There are at least three exemplary choices to this, and the choices can be dependant on the how the Si is made and to what extent with viral capsid is exploited: (1) If one virally templates the Si wire and anneals away the viral scaffold, an SiO2 dielectric layer can be oxidized on as in Approach listage 2; (2) If one virally templates the Si wire and anneal away the viral scaffold, or virally template the Si wire on the outside of the viral capsid, the dielectric layer could be assembled and/or nucleated by specific bifunctional peptides. On one end of the bifunctional peptide can be a peptide that nucleates or binds to the dielectric nanoparticles with controlled size, while the other end can bind to the Si nanowire. An annealing step may follow; (3) If one virally templates the Si wire on the inside of a viral capsid, the outside of the viral capsid can be engineered to bind and/or nucleate the dielectric material, and the formation of the layer proceeds as per normal Belcher p8 wire synthesis.

Stage 3: Formation of Gate Layer (Assemble or Nucteate)

To assemble/nucleate the third layer, the gate material can be a metal. One can use another bifunctional peptide. One end can nucleate or bind the gate material, controlling its thickness by nanoparticle size, while the other end can bind to the dielectric layer. Again at this stage, to create the optimal interface, the gate layer formation can be followed with an annealing step to ensure a good interface between the layers.

For these 3 stages, the modified ends of the wires can be preserved throughout each step.

For each of the two approaches, the assembly can be further bound to other electrode structures, including source and drain structures, using the biological agent comprising binding structures.

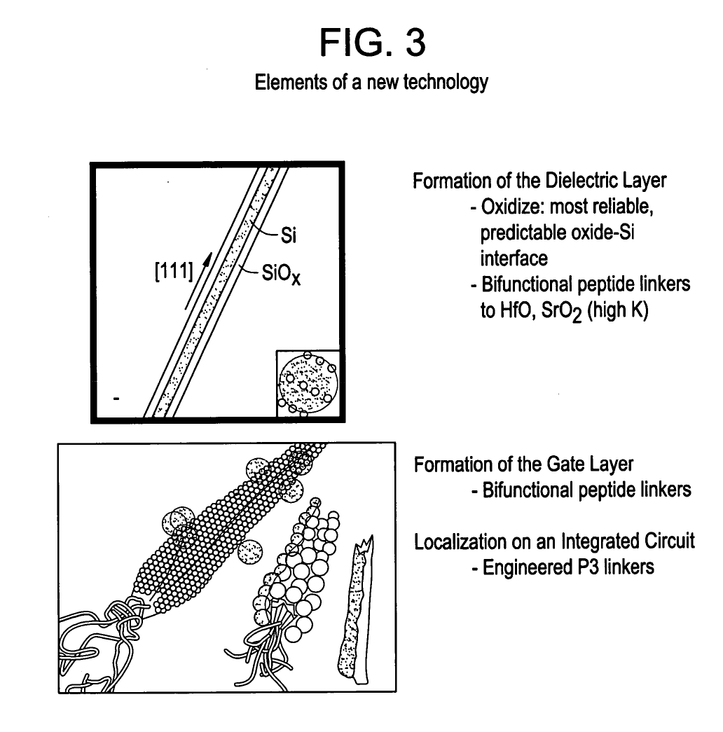

FIG. 3 illustrates in a top view of cross-section of a crystalline silicon nanowire with a silicon dioxide shell. In the bottom view of FIG. 3, a comparable filamentous bacteriophage is illustrated which can be engineered at the end via, for example, p3 engineering, or along the length of the filament via p8 engineering.

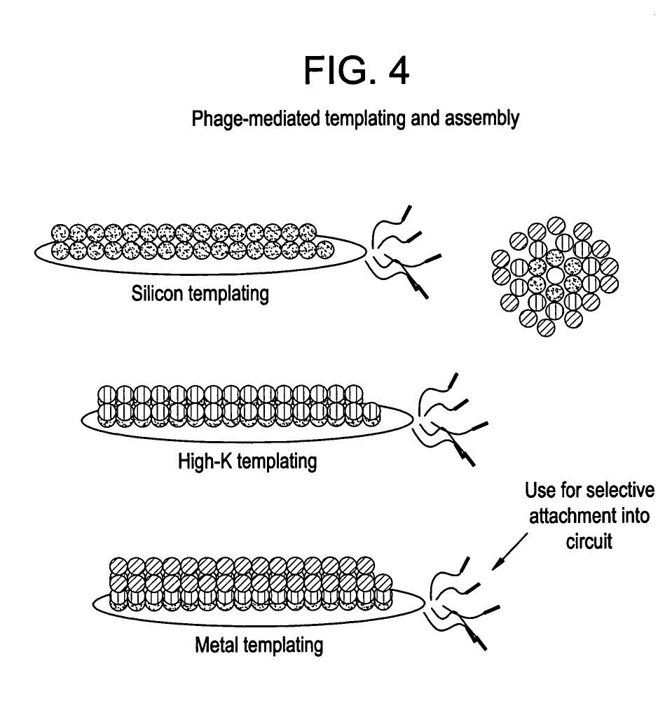

FIG. 4 illustrates phage-mediated templating and assembly. At the top, silicon templating is illustrated to form a nanowire core which can be a channel. In the middle, further templating is illustrated for formation of the high-K dielectric layer surrounding the channel. In the bottom picture, metal templating can be further accomplished to form a gate structure. One or both ends of the virus can be adapted to bind to a substrate which localizes the channel, dielectric, and gate in a useful setting.

All references described herein including patents, patent publications, and journal articles are hereby incorporated by reference in their entirety.

While this invention has been described in reference to illustrative embodiments, the descriptions are not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

1-105. (canceled)

106. An component for use in fabrication of a transistor, the component comprising at least two of the following transistor elements: (i) source, (ii) drain, (iii) channel, (iv) gate, and (v) dielectric, wherein the at least two elements are combined by a biological agent comprising at least two binding structures, wherein each of the binding structures is bound to one of the at least two elements.

107. The component according to claim 106, wherein the transistor is a field effect transistor.

108. The component according to claim 107, wherein the field effect transistor is a MISFET, a MOSFET, a MESFET, or a JFET.

109. The component according to claim 106, wherein the biological agent is a synthetic or engineered peptide.

110. The component of claim 106, wherein the channel comprises a nanowire or nanotube.

111. The component of claim 110, wherein the nanowire comprises a compositionally modulated material.

112. The component of claim 106, wherein the channel comprises an electrically conductive polymer.

113. The component of claim 106, wherein the gate is part of a wrap around gate structure.

114. The component of claim 106, wherein the gate is a metallic gate.

115. The component of claim 106, wherein the gate is a semiconductor gate.

116. The component of claim 106, wherein the dielectric is a high K dielectric.

117. The component of claim 106, wherein the dielectric is an oxide.

118. The component according to claim 106, wherein the source, channel, and drain are present as an integrated structure, and the biological agent comprising at least two binding structures further binds the source and drain to additional source and drain structures.

119. An electronic device comprising a plurality of field effect transistors, wherein the field effect transistors comprise channels comprising nanowires which are substantially monodisperse in length.

120. A nanowire structure comprising a nanowire core and a first nanowire outer layer surrounding the core, wherein a biological agent comprising at least two binding structures is used to combine the nanowire core and the nanowire outer layer.

121. The nanowire structure according to claim 120, further comprising a second nanowire outer layer which surrounds the first outer layer.

122. The nanowire structure of claim 120, wherein the core nanowire is a heterostructure and comprises end materials.

123. An intermediate component for use in fabrication of a transistor, the component comprising at least two of the following transistor elements: (i) source, (ii) drain, (iii) channel, (iv) gate, and (v) dielectric, wherein the at least two elements are combined by a biological agent comprising at least two binding structures, wherein each of the binding structures is bound to one of the at least two elements.

124. The intermediate component according to claim 123, wherein the transistor is a field effect transistor comprising a wrap around gate.

125. A process for fabricating elements of a field effect transistor, comprising the step of combining at least a channel element and a source or drain element, wherein at least one biological agent comprising at least two binding structures is used to combine the channel element and the source or drain element.

126. A method for engineering the surface of a nanowire with an outer layer material comprising the step of binding the surface of the nanowire with a biological agent comprising at least two binding structures, one binding structure for the surface of the nanowire, and one binding structure for the outer layer material.

127. The method of claim 126, wherein the nanowire is formed using biological agents.

128. The method of claim 126, wherein a material is deposited on the surface of the outer layer material.

129. A biological agent represented by A-B-C, wherein A and C are selective binding structures and B is an optional linking structure, wherein A and C selectively bind to a channel, a dielectric, a gate, a source, or a drain material.

130. A field effect transistor comprising a nanowire or nanotube channel, a dielectric material surrounding the channel, and a metal or semiconductor layer surrounding the dielectric material.

131. The transistor according to claim 130, wherein the dielectric material or metal layer surrounding the dielectric material are deposited using biological agents.

132. The field effect transistor according to claim 130, wherein the nanowire or nanotube forms an integrated structure with source and drain structures.

133. A method of forming a field effect transistor comprising
providing a nanowire or nanotube channel,
depositing a dielectric material to surround the channel, and
depositing a metal layer or semiconductor layer to surround the dielectric material, wherein at least one of the deposition steps is performed using a biological agent.

134. A transistor comprising a nanowire or nanotube channel, a dielectric material surround the channel and having a K value of about 10 or more, and a gate layer surrounding the dielectric material.

135. A method of forming a dielectric layer surrounding a nanowire or a nanotube comprising the steps of providing the nanowire or nanotube, providing the dielectric material or a precursor thereof, providing a biological agent which comprises at least two binding structures, and forming the dielectric layer on the nanowire or nanotube in the presence of the biological agent.

136. A method of forming a gate layer surrounding a dielectric material comprising the steps of providing the dielectric material, providing the gate material or a precursor thereof, providing a biological agent which comprises at least two binding structures, and forming the gate layer on the dielectric material in the presence of the biological agent.

137. A method of forming a connection between a nanowire or a nanotube and a source or a drain, comprising the steps of providing a biological agent which comprises at least two binding structures, providing the nanowire or the nanotube, providing the source or drain, and connecting the nanowire or nanotube with the source or drain in the presence of the biological agent.

138. A method of forming a metal layer surrounding a nanowire or a nanotube comprising the steps of providing the nanowire or nanotube, providing the metal material or a precursor thereof, providing a biological agent which comprises at least two binding structures, and forming the metal layer on the nanowire or nanotube in the presence of the biological agent.

139. A method of forming a semiconductor layer surrounding a nanowire or a nanotube comprising the steps of providing the nanowire or nanotube, providing the semiconductor material or a precursor thereof, providing a biological agent which comprises at least two binding structures, and forming the semiconductor layer on the nanowire or nanotube in the presence of the biological agent.
